(19) United States
(12) Patent Application Publication
(10) Pub. No.: US 2024/0311531 A1
(43) Pub. Date: Sep. 19, 2024

(54) A METHOD AND A SYSTEM FOR UV PROTECTION PREDICTION OF A SUNSCREEN PRODUCT

(71) Applicants: BASF SE, Ludwigshafen am Rhein (DE); THE CHANCELLOR, MASTERS AND SCHOLARS OF THE UNIVERSITY OF CAMBRIDGE, Cambridge (GB)

(72) Inventors: Dong Ryeol LEE, Hong Kong (CN); Jun Tao XIA, Shanghai (CN); Myriam SOHN, Grenzach-Wyhlen (DE); Bernd HERZOG, Grenzach-Wyhlen (DE); Liwei CAO, Cambridge (GB); Alexei LAPKIN, Cambridge (GB)

(21) Appl. No.: 18/273,285

(22) PCT Filed: Jan. 5, 2022
(86) PCT No.: PCT/EP2022/050120

§ 371 Date: Jul. 20, 2023

(30) Foreign Application Priority Data
Jan. 21, 2021 (WO) PCT/CN2021/073129

(51) Int. Cl. G06F 30/27

(52) U.S. Cl. CPC G06F 30/27

(57) ABSTRACT

The present invention relates to a method and a system for UV protection prediction of a sunscreen product, comprising a) selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter; c) inputting the features into a predictive model, which is built and fitted by using one or more machine learning techniques; and d) calculating the UV protection prediction value of the sunscreen product by the predictive model of step c).

### TECHNICAL FIELD

The present invention relates to a method and a system for UV protection prediction of a sunscreen product, comprising a) selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter; c) inputting the features into a predictive model, which is built and fitted by using one or more machine learning techniques; and d) calculating the UV protection prediction value of the sunscreen product by the predictive model of step c).

### BACKGROUND ART

In sun care application development, the performance evaluation is challenging because it is mandatory to evaluate with SPF and UVA-PF test protocols in vivo. These tests cost a lot in the development phase and are time-consuming as the tests are in vivo. To deal with this challenge, there have been trials to predict the UV protection performance by various modelling. However, in reality the prediction accuracy was very much limited by the UV combination based models because the real UV protection performance is determined not only by UV filters combination, but also other formulation ingredients, such as emollients, emulsifiers, polymers. Besides, formulation texture types such Water-in-Oil, Oil-in-Water, Spray, Lotion types all impact on the UV protection performance. Another approach was to evaluate the performance with in vitro tests. Approaches have been intensively investigated to validate the corresponding correlation between the value with in vivo and the one with in vitro. However, there have been still gaps between the in vivo and the in vitro values for both SPF and UVA-PF. Therefore, the use of the in vitro test was not well accepted as prediction tool.

Jiyong Shim, Jun Man Lim, Sun Gyoo Park, Machine learning for the prediction of sunscreen sun protection factor and protection grade of UVA, Experimental Dermatology, 2019, Volume 28, Issue 7, Pages: 872-874 reported a prediction model for sunscreen sun protection factor (SPF) and protection grade of ultraviolet (UV) A (PA) based on machine learning, in which the concentration of UV filter substances as well as four additional factors, such as presence of pigment, concentration of pigment grade titanium dioxide, type of formulation and type of product, were used for the prediction model. However, the prediction accuracy and efficiency were still not very satisfactory.

### SUMMARY OF INVENTION

It is therefore an object of the present invention to overcome the drawback in the art.

According to one aspect of the present invention, said object can be achieved by a method for UV protection prediction of a sunscreen product, the method comprising the following steps:
a) selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter;
c) inputting the features into a predictive model, which is built and fitted by using one or more machine learning techniques; and
d) calculating the UV protection prediction value of the sunscreen product by the predictive model of step c).

In accordance with an embodiment of the method according to the present invention, the method can optionally further comprise a step b) for transforming the features of step a) by performing one or more dimensionality reduction techniques, between step a) and step c).

According to another aspect of the present invention, said object can be achieved by a system for UV protection prediction of a sunscreen product, the system including the following modules:
a) a module for selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter;
c) a module for inputting the features into a predictive model, which is built and fitted by using one or more machine learning techniques; and
d) a module for calculating the UV protection prediction value of the sunscreen product by the predictive model of module c).

In accordance with an embodiment of the system according to the present invention, the system can optionally further include a module b) for transforming the features of module a) by performing one or more dimensionality reduction techniques.

### BRIEF DESCRIPTION OF DRAWINGS

Each aspect of the present invention will be illustrated in more detail in conjunction with the accompanying drawings, wherein

FIG. 1 shows the correlation between the inputs of an illustrative dataset;

FIG. 2 shows the correlation between the inputs and the outputs in the illustrative dataset;

FIG. 3 shows the feature importances in PCA analysis of the O/W type sunscreen products in the illustrative dataset;

FIG. 4 shows the feature importances in PCA analysis of the gel type sunscreen products in the illustrative dataset;

FIG. 5 shows the feature importances in PCA analysis of all type sunscreen products in the illustrative dataset;

FIG. 6 shows the correlations between Bayesian regression model prediction and experimental results of the O/W type sunscreen products in the illustrative dataset: (a) SPF predicted by machine learning and SPF observed in vivo, and (b) UVA predicted by machine learning and SPF observed in vitro; and

FIG. 7 shows the correlations between Bayesian regression model prediction and experimental results of the gel type sunscreen products in the illustrative dataset: (a) SPF predicted by machine learning and SPF observed in vivo, and (b) UVA predicted by machine learning and SPF observed in vitro.

### DETAILED DESCRIPTION OF PREFERRED EMBODIMENTS

All publications, patent applications, patents and other references mentioned herein, if not otherwise indicated, are explicitly incorporated by reference herein in their entirety for all purposes as if fully set forth.

Unless otherwise defined, all technical and scientific terms used herein have the same meaning as commonly understood by one of ordinary skill in the art to which this invention belongs. In case of conflict, the present specification, including definitions, will control.

When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

The present invention, according to one aspect, relates to a method for UV protection prediction of a sunscreen product, the method comprising the following steps:
a) selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter;
c) inputting the features into a predictive model, which is built and fitted by using one or more machine learning techniques; and
d) calculating the UV protection prediction value of the sunscreen product by the predictive model of step c).

In accordance with an embodiment of the method according to the present invention, the method can optionally further comprise a step b) for transforming the features of step a) by performing one or more dimensionality reduction techniques, between step a) and step c).

### a) Selecting the Features of the Sunscreen Product

In accordance with another embodiment of the method according to the present invention, in step a), the features of the sunscreen product can be selected, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter.

After extensive research, the inventors of the present invention have noticed the correlation between the UV absorption spectrum behavior, for example the wavelength of the maximum absorbance (λmax) and molar absorptivity (ε) at 308 nm, of UVB sunscreen filter in emollients and their solubility parameter (Seong-Rae Kim, Seung-Ki Lee, Choon-Koo Zhoh, Effect of Emollients on the UV Absorption Behavior of Ethylhexyl Methoxycinnamate, J. Kor. Soc. Esthe. & Cosm. Vol. 7, No. 1 (2012) p. 9-16), and further found that the solubility property of the emollients can be impacted by their polarity, and revealed that the difference in the polarity of the emollients standardly used in sunscreens leads to a difference in the absorbance of the UV filters, and an increase in the emollient polarity leads to an increase in the UVA protection factor value (Myriam Sohn, Lola Amorós-Galicia, Stanislaw Krus, Karine Martin, Bernd Herzog, Effect of emollients on UV filter absorbance and sunscreen efficiency, Journal of Photochemistry & Photobiology, B: Biology 205 (2020) 111818). It can be hypothesized that there would be correlations between the polarity of emollients and the SPF values.

The polarity of emollients can be determined by the oil/water interfacial tension (IFT), which is a preferred parameter in the emulsification process, and can be easily measured by the pendant drop method, and then classified into three groups:
Low Polarity: IFT≥35 mN/m,
Medium Polarity: 25 mN/m≤IFT<35 mN/M,
High Polarity: IFT<25 mN/m.

The polarity classification of some commercial emollients is summarized in Table 1.

TABLE 1
Polarity classification of some commercial emollients.

| Emollient trade name | INCI Name | Polarity |
|---|---|---|
| Myritol ® 331 | Cocoglycerides | high |
| Cetiol ® B | Dibutyl Adipate | high |
| Cetiol ® AB | C12-C15 Alkyl Benzoate | high |
| Eutanol ® G | Octyldodecanol | high |
| Cetiol ® SB 45 | Butyrospermum Parkii Shea Butter) | high |
| Cetiol ® PGL | Hexyldecanol (and) Hexyldecyl Laurate | high |
| Myritol ® 312 | Capric/Caprylic Triglyceride | medium |
| Cetiol ® J 600 | Oleyl Erucate | medium |
| Cegesoft ® PS 6 | Vegetable Oil | medium |
| Cetiol ® SN | Cetearyl Isononanoate | medium |
| Cetiol ® LC | Coco-Caprylate/Caprate | medium |
| Cetiol ® V | Decyl Oleate | medium |
| Cetiol ® A | Hexyl Laurate | medium |
| Cetiol ® C 5C | Coco-Caprylate/Caprate | medium |
| Cetiol ® RLF | Caprylyl-Caprylate/Caprate | medium |
| Cetiol ® CC | Dicaprylyl Carbonate | medium |
| Cetiol ® Sensoft | Propylheptyl Caprylate | medium |
| Cetiol ® OE | Dicaprylyl Ether | low |
| Luvitol ® Lite | Hydrogenated Polyisobutene | low |
| Cetiol ® Ultimate | Undecane (and) Tridecane | low |

Moreover, the inventors of the present invention have also found that the balanced UV protection in oil phase and in water phase would be important to achieve a high SPF in addition to the balanced protection in UVA and UVB range.

It would be too complicated to consider and quantify all these formulation parameters and impacts in the machine learning for the SPF prediction. The prediction methods in the prior art never include as many as nine features, let alone the above specific nine features. For example, Shim's machine learning method only includes the concentration of UV filter substances as well as presence of pigment, concentration of pigment grade titanium dioxide, and type of formulation and type of product. In contrast, the above nine features can be taken into consideration according to the present invention, and therefore, the performance of the sunscreen products can be reflected more comprehensively, and the prediction results obtained accordingly are much more accurate.

An illustrative dataset for the O/W type sunscreen products is summarized in Table 2. For example, the dataset can be split at a ratio of 7:1:2 for training, validation and test respectively.

TABLE 2
An illustrative dataset.

| | | | Inputs | | | | | | | | |
|---|---|---|---|---|---|---|---|---|---|---|---|
| | | | | | | | Scattering/ | UV | | | |
| | Outputs | | | | | | Absorber | filter | | | |
| | SPF | UVA-PF | Visocosity | Emollient | Emollient | Emollient | in UV | in | UVA/ | UVB | UVA |
| | in_vivo | in_vitro | (mPa*s) | high | low | medium | filters | water | UVB | filter | filter |
| count | 131 | 131 | 131 | 131 | 131 | 131 | 131 | 131 | 131 | 131 | 131 |
| mean | 43.78 | 17.59 | 18447.56 | 21.40 | 12.69 | 2.81 | 0.28 | 0.37 | 0.90 | 6.46 | 5.13 |
| std | 16.91 | 6.73 | 18221.39 | 87.45 | 90.70 | 4.63 | 0.28 | 0.24 | 0.69 | 4.37 | 3.16 |
| min | 9.00 | 6.20 | 200.00 | 0.00 | 0.00 | 0.00 | 0.00 | 0.00 | 0.00 | 0.00 | 0.00 |
| 25% | 31.00 | 11.50 | 5750.00 | 8.00 | 2.00 | 0.00 | 0.00 | 0.22 | 0.50 | 3.00 | 3.50 |
| 50% | 45.00 | 17.70 | 12000.00 | 14.00 | 4.00 | 0.00 | 0.28 | 0.38 | 0.83 | 6.00 | 5.00 |
| 75% | 57.00 | 22.30 | 26000.00 | 19.00 | 5.00 | 4.00 | 0.46 | 0.50 | 1.25 | 10.00 | 8.00 |
| max | 90.00 | 38.60 | 105000.00 | 1010.00 | 1042.00 | 19.00 | 1.00 | 0.84 | 3.50 | 21.00 | 10.00 |

Feature visualization would be helpful for comprehensive understanding of the features. However, for a high dimensional feature space, it would not be an easy task to visualize the feature space clearly. Instead of visualizing the feature space at one time, it would be recommended to analyse the pairwise correlation by exploratory data analytics (EDA), in which calculation correlation matrix is one of the most common techniques. The correlation matrix denoted by r is a square matrix based on Pearson product-moment correlation coefficients, which can be used as a measure of the linear correlation between the inputs and the outputs in the dataset:

$${r}_{x , y} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} ,$$

where n is the size of the feature, xᵢ, yᵢ are the individual points indexed with i; and x̅ is the mean of the feature x (x̅=Σᵢ₌₁ⁿxᵢ) and analogously for y̅.

Pearson's r value ranges from +1 to −1. A value of 0 indicates that there is no association between the two variables x and y. A value greater than 0 indicates a positive association; that means the value of one variable increases, as the value of the other variable increases. A value less than 0 indicates a negative association; that means the value of one variable increases, as the value of the other variable decreases. EDA based on Pearson's r value would be helpful to get some basic insights about the linear correlations between the outputs and the input features. Thus, the features that are relatively highly related to the outputs can be selected as the “exploratory feature” for further machine learning model construction.

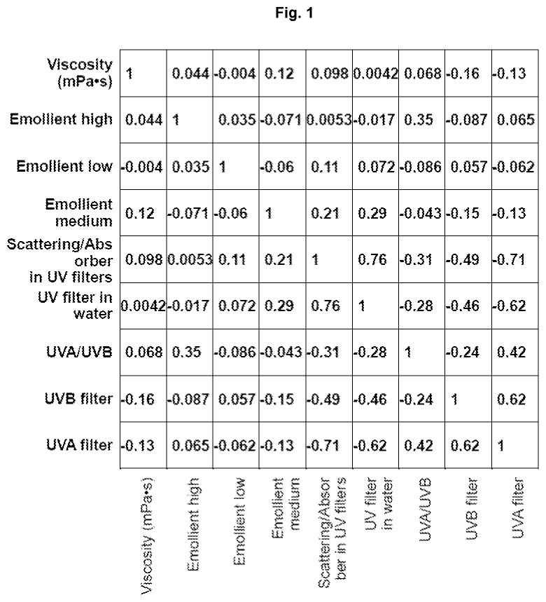

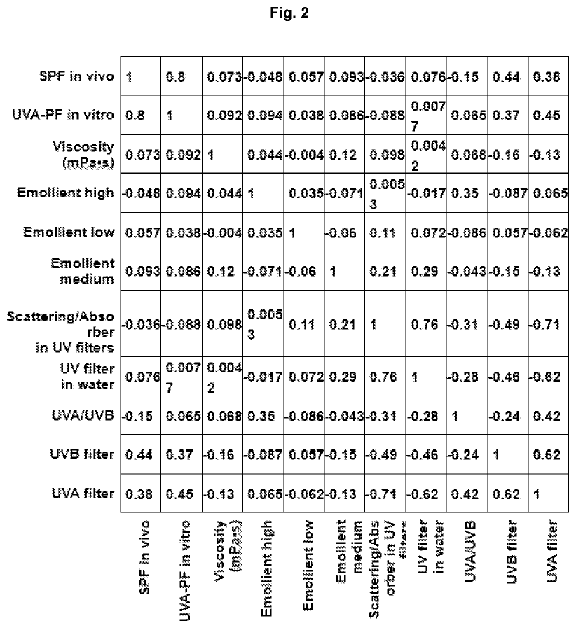

FIG. 1 shows the correlation between the inputs in the illustrative dataset; and FIG. 2 shows the correlation between the inputs and the outputs in the illustrative dataset. It can be seen that the correlation between the input parameters is not large, while the correlation between the two outputs is strong.

When the datasets are high dimensional, it would always be recommended to perform dimensionality reduction by feature selection. The basic idea of feature selection is to evaluate the importance of each feature to the output, and remove the features having less influence on the performance of machine learning models, and only keep the features having more influence on the machine learning models. The features selected are usually different for different machine learning models. Random forest algorithm can be used as an example to show how feature selection works. Random forest can be constructed with individual decision tree models. The input can be represented as the node and the possible outcome as the edges.

Feature importance can be calculated as the decrease in node impurity weighted by the probability of reaching that node. For each decision tree within random forest model, the node importance can be calculated using Gani Importance which is shown below:

$${NI}_{j} = {w}_{j} {C}_{j} - {w}_{left ( j )} {C}_{left ( j )} - {w}_{right ( j )} {C}_{right ( j )} ,$$

where NIⱼ is the importance of node j, wⱼ is the weighted number of samples reaching node j, Cⱼ is the impurity value of node j, left(j) is the child node from right split on node j, and right(j) is the child node from right split on node j.

The importance for each feature on a decision tree can be calculated as:

$${FI}_{i} = \frac{{\sum }_{j : node j splits on feature i} {NI}_{j}}{{\sum }_{k ∈ all nodes} {NI}_{k}} ,$$

where FIᵢ is the feature importance of feature i, NIⱼ is the importance of node j.

These can then be normalized to a value between 0 and 1 by dividing by the sum of all feature importance values:

$${normFI}_{i} = \frac{{FI}_{i}}{{\sum }_{j ∈ all features} {FI}_{j}} .$$

The final feature importance, for the random forest model, is the average over all the decision trees. The expression is indicated as follow:

$${RF}_{i} = \frac{{\sum }_{j ∈ all trees} {normFI}_{ij}}{T} ,$$

where RFᵢ is the importance of feature i calculated from all decision trees in the RF model, normFIᵢⱼ is the normalized feature importance for feature i in tree j, and T is the total number of the decision trees.

The importance of the features of the sunscreen products in the illustrative dataset is summarized in Table 3.

TABLE 3
The importance of the features of the sunscreen
products in the illustrative dataset.

| Feature | Importance |
|---|---|
| Viscosity (mPa · s) | 0.34 |
| Emollient high | 0.17 |
| Emollient low | 0.01 |
| Emollient medium | 0.02 |
| Scattering/Absorber in UV filters | 0.05 |
| UV filter in water | 0.12 |
| UVA/UVB | 0.08 |
| UV_B_filter | 0.11 |
| UV_A_filter | 0.10 |

### b) Transforming the Features of Step a)

In accordance with another embodiment of the method according to the present invention, the method can optionally further comprise a step b) for transforming the features of step a) by performing one or more dimensionality reduction techniques, between step a) and step c).

In accordance with another embodiment of the method according to the present invention, in step b), one or more dimensionality reduction techniques selected from the group consisting of Principal Component Analysis (PCA), Linear Discriminant Analysis (LDA), and Kernel Principal Component Analysis (KPCA) can be performed.

Feature extraction can be used for dimensionality reduction to create a new feature space by projecting the original feature space with certain rules. Principal component analysis (PCA) can be selected as an illustrative example of feature extraction. Implementation of other feature extraction techniques, such as linear discriminate analysis and kernel principal component analysis, can be done in a similar manner.

PCA can be used to find the principal component of the features in the sense that the covariance between such component and outputs are largest. In PCA, a D×K dimensional transformation matrix W is constructed to convert the original feature space x=[x¹, x², . . . , x^{D}] into a new feature space \hat{x}=[x¹, x², . . . , x^{K}] to facilitate further analysis. Usually, the transformation matrix is constructed based on the covariance matrix between different features. The covariance between features xⁱ and x^{j} can be calculated as:

$${σ}_{ij} = \frac{1}{n} {\sum }_{k = 1}^{n} ( {x}_{k}^{i} - \overline{{x}^{i}} ) ( {x}_{k}^{j} - \overline{{x}^{j}} ) .$$

Based on such covariance definition, a D×D dimensional covariance matrix from feature space x=[x¹, x², . . . , x^{D}] can be obtained then by choosing the K largest eigenvalues and the corresponding eigenvectors, the transformation matrix could be constructed. In such a framework, the feature importance is defined as the ratio between its corresponding eigenvalue and the overall sum of all eigenvalues.

$$\frac{{λ}_{i}}{{\sum }_{i = 1}^{D} {λ}_{i}} .$$

Detailed information of the experimental procedures of Principal Component Analysis (PCA), Linear Discriminant Analysis (LDA), and Kernel Principal Component Analysis (KPCA) can be found, for example, in Sebastian Raschka, Python Machine Learning, 2nd Edition, Packt Publishing, 2017. Especially detailed information of Principal Component Analysis (PCA) can be found, for example, in I. T. Jolliffe, Principal Component Analysis, 2nd Edition, Springer, 2002. For example, scikit-learn (sklearn) is an open source machine learning library which provides various tools for model fitting, data preprocessing, model selection and evaluation. PCT can be implemented by sklearn.

In accordance with another embodiment of the method according to the present invention, in step b), Principal Component Analysis (PCA) can be performed to obtain four principal components of the features.

The inventors of the present invention have found that four principal components can represent more than 85% of the information of the entire feature space. Moreover, the inventors of the present invention have also fitted the Bayesian regression model with three, four, five, and six principal components respectively, and found the lowest testing error in the model with four principal components.

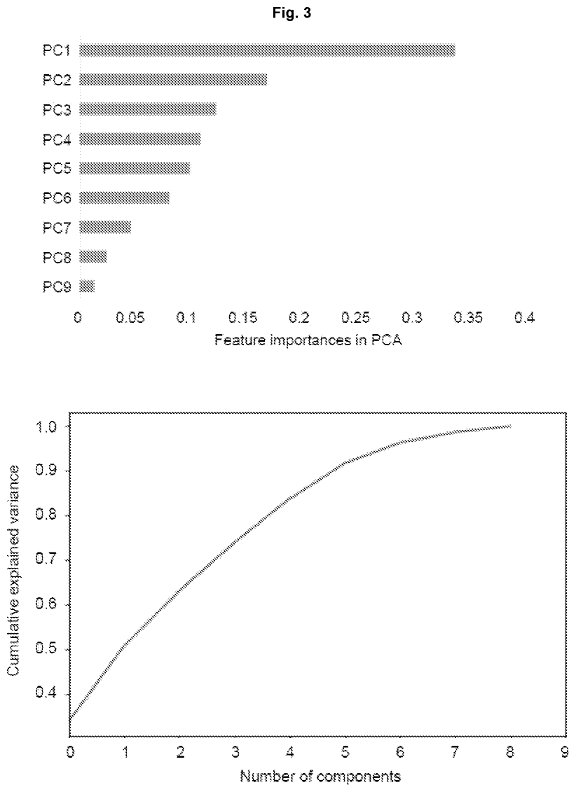

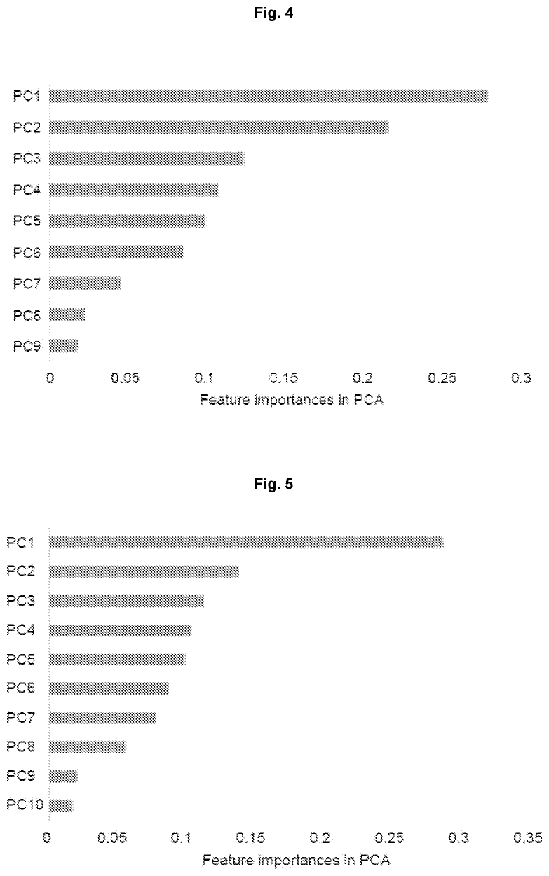

FIG. 3 shows the feature importances in PCA analysis of the O/W type sunscreen products in the illustrative dataset; FIG. 4 shows the feature importances in PCA analysis of the gel type sunscreen products in the illustrative dataset; and FIG. 5 shows the feature importances in PCA analysis of all type sunscreen products in the illustrative dataset.

c) Inputting the Features into a Predictive Model

In accordance with another embodiment of the method according to the present invention, in step c), the features can be inputted into a predictive model, which is built and fitted by using one or more machine learning techniques.

In accordance with another embodiment of the method according to the present invention, in step c), the predictive model can be built and fitted by using one or more machine learning techniques selected from the group consisting of Ridge Regression, Bayesian Regression, Supporting Vector Machine (SVM), k-Nearest Neighbours (k-NN) Regression, Decision Tree, and Gaussian Process Regression.

Ridge regression can be used as a linear model in which the target value is expected to be a linear combination of features,

$$\hat{y} ( ω , x ) = {ω}_{0} + {ω}_{1} {x}_{1} + … + {ω}_{n} {x}_{n} .$$

In the ridge regressor, the ridge coefficients minimize a penalized residual sum of squares:

$${min}_{w} {‖ X ω - y ‖}_{2}^{2} + α {‖ ω ‖}_{2}^{2} ,$$

where w is the coefficient parameters, α≥0 is a shrinkage coefficient, the larger α it is, the greater the amount of shrinkage and thus the coefficients become more robust to collinearity. α can be tested from 10⁻³ to 10³, and an optimal value can be selected for the model fitting.

SVM regression can be used to perform linear regression in the high-dimension feature space using ϵ-insensitive loss, and tries to reduce model complexity by minimizing ∥ω∥². This can be described by introducing (non-negative) slack variables ξᵢ, ξᵢ*, i=1, . . . , n, to measure the deviation of training samples outside ϵ-insensitive zone. Thus, the SVM regression can be formulated as minimization of the following function:

$$\begin{matrix}min \frac{1}{2} ‖ ω ‖ + C {\sum }_{i = 1}^{n} ( {ξ}_{i} + {ξ}_{i}^{*} ) , \\ s . t . {y}_{i} - f ( {x}_{i} , ω ) \leq ϵ + {ξ}_{i}^{*} , \\ f ( {x}_{i} , ω ) - {y}_{i} \leq ϵ + {ξ}_{i} , \\ {ξ}_{i} , {ξ}_{i}^{*} \geq 0 , i = i , … , n .\end{matrix}$$

This optimization problem can be transformed into the dual problem and its solution is given by

$$\begin{matrix}f ( x ) = {\sum }_{i = 1}^{{n}_{sv}} ( {α}_{i} - {α}_{i}^{*} ) K ( {x}_{i} , x ) , \\ s . t . 0 \leq {α}_{i} \leq C , 0 \leq {α}_{i}^{*} \leq C ,\end{matrix}$$

where nₛᵥ is the number of Support Vectors (SVs), and K is the kernel function

$$K ( x , {x}_{i} ) = {\sum }_{j = 1}^{m} {g}_{j} ( x ) {g}_{j} ( {x}_{i} ) .$$

Three different types of kernels can be tested, namely linear, RBF, polynomial kernel. For the RBF kernel, parameter γ and C can be tested within range of [10⁻³, 10³], and for the polynomial kernel, another parameter degree can be varied between 1 and 10.

k-NN regressor uses feature similarity to predict values of the new data points, which means that the new point is assigned a value based on how closely it resembles the points in the training dataset. Euclidean distance can be selected for the distance calculation. The key parameter which define the number of n-neighbours can be tested within range of 1 to 100.

Decision tree can be used as a supervised machine learning model to predict a target by learning decision rules from features. A decision tree can be constructed by recursive partitioning, which means that the tree can split from the root node, and each node can be split into left and right child node. A maximal depth of the tree can be set as a limit when a decision tree is pruned. In order to split the nodes at the most informative features, the objective function can be used to maximize the information gain at each split, which is defined as follow:

$$IG ( {D}_{P} , f ) = I ( {D}_{p} ) - ( \frac{{N}_{left}}{{N}_{p}} I ( {D}_{left} ) + \frac{{N}_{l right}}{{N}_{p}} I ( {D}_{right} ) ) ,$$

where f is the feature to perform the split, D_{P}, D_{left} and D_{right} are the datasets of the parent and child nodes, I is the impurity measure, Np is the total number of samples at the parent node, and N_{left} and N_{right} are the number of samples in the child nodes. For Decision Tree, different values of n can be between 5 and 15, where n is the number such that impure nodes must have n or more observations to be split, can be tested at a tree fitting stage. The best level of pruning can be evaluated through crossvalidation (where the best level is the one that produced the smallest tree that is within one standard error of the minimum-cost subtree).

Gaussian process regressor can be used as a nonparametric kernel-based probabilistic model which is based on Gaussian processes (GP). The prior of the GP needs to be specified based on the training dataset, and the prior's covariance can be specified by passing a kernel object. The hyperparameters of the kernel then are optimized based on maximizing the lag-marginal-likelihood, given by

$$log p ( y \vert x , θ ) = - \frac{1}{2} {y}^{T} {K}^{- 1} y - \frac{1}{2} log \vert K \vert - \frac{n}{2} log 2 π ,$$

where K is the covariance matrix, θ is the vector of hyperparameter, n is the number of data points.

For example, Ridge Regression, Supporting Vector Machine (SVM), k-Nearest Neighbours (k-NN) Regression, Decision Tree, and Gaussian Process Regression can be implemented by sklearn.

In accordance with another embodiment of the method according to the present invention, in step c), the predictive model can be built and fitted by using Bayesian regression.

Bayesian regressor can be used to evaluate a probabilistic model of the regression problem. The parameters ω, α and λ can be evaluated jointly when fitting the model. The prior for the coefficient ω is given by a spherical Gaussian distribution:

$$p ( ω \vert λ ) = 𝒩 ( ω \vert 0 , {λ}^{- 1} {I}_{p} ) ,$$

the priors over α and λ are selected to be gamma distributions, the conjugate prior for precision of the Gaussian distribution. The regularization parameters α and λ can be evaluated by maximizing the log marginal likelihood.

Detailed information of the experimental procedures of Bayesian regression model can be found, for example, in Christopher M. Bishop, Pattern Recognition and Machine Learning, Springer, 2006. For example, Bayesian Regression can also be implemented by sklearn.

In accordance with another embodiment of the method according to the present invention, in step c), hyperparameter tuning can be performed for the predictive model.

For example, in case of Bayesian regression model, two hyperparameters αᵢₙᵢ and λᵢₙᵢₜ need to be set for giving the initial value of the maximization procedure. Furthermore, there are four more hyperparameters, α₁, α₂, λ₁ and λ₂, which are the parameters for the gamma prior distributions over α and λ. α₁ and λ₁ are the shape parameter for the Gamma distribution prior over α and λ. α₂ and λ₂ are the inverse scale parameter (rate parameter) for the Gamma distribution prior over α and λ.

The dataset can be split into a training, a validation and a test dataset. The grid search approach can be applied for exhaustively sampling the hyperparameter space in order to obtain the optimized hyperparameter. For example, the grid can be set as follows:

```
# Define our parameters to run a grid search over
param_grid = {
  “alpha_init”: np.logspace(−2, 2, 10),
  “lambda_init”: np.logspace(−2, 2, 10),
  “alpha_1”: np.logspace(−6, 2, 20),
  “alpha_2”: np.logspace(−6, 2, 20),
  “lambda_1”: np.logspace(−6, 2, 20),
  “lambda_2”: np.logspace(−6, 2, 20),
}
```

The parameters combination which gives the lowest mean square error in the validation dataset can be selected as the hyperparameters for the model.

In accordance with another embodiment of the method according to the present invention, in step c), the predictive model can be fitted by a dataset of the features including a formulation type, a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, a ratio of absorbing type UV filter vs scattering/reflecting type UV filter, a SPF in vivo and a UVA-PF in vitro.

### d) Calculating the UV Protection Prediction Value of the Sunscreen Product

In accordance with another embodiment of the method according to the present invention, in step d), the UV protection prediction value of the sunscreen product can be calculated by the predictive model of step c).

In accordance with another embodiment of the method according to the present invention, in step d), the UV protection prediction value can be selected from the group consisting of SPF in vivo and UVA-PF in vitro.

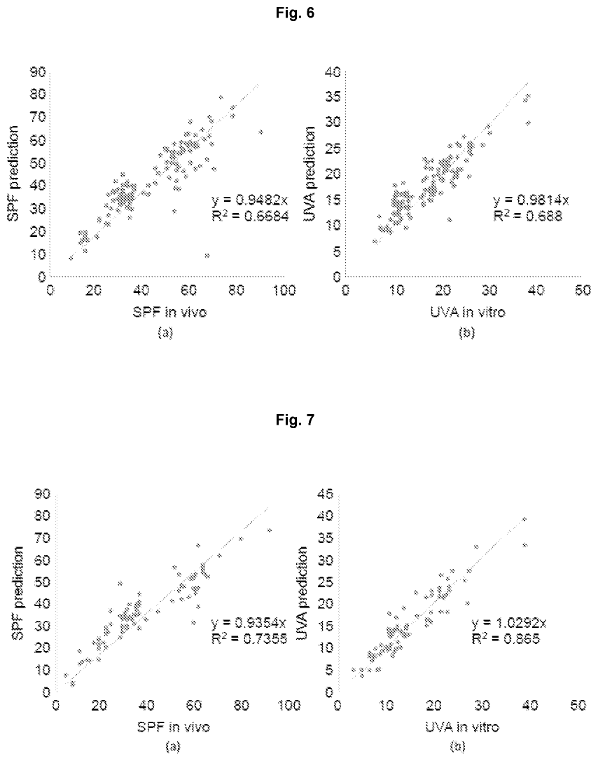

FIG. 6 shows the correlations between Bayesian regression model prediction and experimental results of the O/W type sunscreen products in the illustrative dataset: (a) SPF predicted by machine learning and SPF observed in vivo, and (b) UVA predicted by machine learning and SPF observed in vitro; and FIG. 7 shows the correlations between Bayesian regression model prediction and experimental results of the gel type sunscreen products in the illustrative dataset: (a) SPF predicted by machine learning and SPF observed in vivo, and (b) UVA predicted by machine learning and SPF observed in vitro.

Relative prediction errors can also be calculated as follow to evaluate the accuracy of the predictive models

$$\frac{\vert output - prediction \vert}{output} \times 100 \% .$$

where output is the experimental results in the dataset, and prediction is prediction value calculated by the predictive models.

Relative prediction errors of the six predictive models without PCA for the O/W and gel type sunscreen products are summarized in Tables 4 and 5 respectively, in which Bayesian Regression is preferred both for the O/W and gel type sunscreen products.

TABLE 4
Relative prediction errors for the O/W type sunscreen products.

| | | | | | |
|---|---|---|---|---|---|
| Ridge | Bayesian | | | Decision | |
| Regression | Regression | SVM | k-NN | Tree | GP |

SPF in vivo prediction

| | | | | | |
|---|---|---|---|---|---|
| 26.85 | 18.10 | 18.29 | 19.51 | 19.77 | 28.97 |

UVA-PF in vitro prediction

| | | | | | |
|---|---|---|---|---|---|
| 36.28 | 15.64 | 16.89 | 20.32 | 18.83 | 45.99 |

TABLE 5
Relative prediction errors for the gel type sunscreen products.

| | | | | | |
|---|---|---|---|---|---|
| Ridge | Bayesian | | | Decision | |
| Regression | Regression | SVM | k-NN | Tree | GP |

SPF in vivo prediction

| | | | | | |
|---|---|---|---|---|---|
| 31.25 | 20.32 | 22.71 | 28.05 | 24.91 | 30.77 |

UVA-PF in vitro prediction

| | | | | | |
|---|---|---|---|---|---|
| 33.38 | 16.87 | 18.72 | 19.87 | 18.42 | 41.89 |

Relative prediction errors of Bayesian Regression without and with 6 PCA, 5 PCA, 4 PCA, and 3 PCA for the O/W type sunscreen products are summarized in Table 6, in which Bayesian Regression with 4 PCA is preferred for the lowest prediction errors.

TABLE 6
Relative prediction errors of Bayesian
Regression without and with PCA.

| | | | | | |
|---|---|---|---|---|---|
| Model fitting | without PCA | 6 PCA | 5 PCA | 4 PCA | 3 PCA |

SPF in vivo prediction

| | | | | | |
|---|---|---|---|---|---|
| Prediction error | 18.10 | 16.76 | 16.13 | 14.97 | 15.44 |

UVA-PF in vitro prediction

| | | | | | |
|---|---|---|---|---|---|
| Prediction error | 15.64 | 15.65 | 14.28 | 9.25 | 10.07 |

The present invention, according to another aspect, relates to a system for UV protection prediction of a sunscreen product, the system including the following modules:
a) a module for selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter;
c) a module for inputting the features into a predictive model, which is built and fitted by using one or more machine learning techniques; and
d) a module for calculating the UV protection prediction value of the sunscreen product by the predictive model of module c).

In accordance with an embodiment of the system according to the present invention, the system can optionally further include a module b) for transforming the features of module a) by performing one or more dimensionality reduction techniques.

### a) Module for Selecting the Features of the Sunscreen Product

In accordance with another embodiment of the system according to the present invention, in module a), the features of the sunscreen product can be selected, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter.

The technical details for the illustrative dataset and the importance and correlation analysis of the features can be found in step a) of the method according to the present invention.

### b) Module for Transforming the Features of Module a)

In accordance with another embodiment of the system according to the present invention, the system can optionally further include a module b) for transforming the features of module a) by performing one or more dimensionality reduction techniques.

In accordance with another embodiment of the method according to the present invention, in module b), one or more dimensionality reduction techniques selected from the group consisting of Principal Component Analysis (PCA), Linear Discriminant Analysis (LDA), and Kernel Principal Component Analysis (KPCA) can be performed.

In accordance with another embodiment of the method according to the present invention, in module b), Principal Component Analysis (PCA) can be performed to obtain four principal components of the features.

The technical details for the feature extraction, for example Principal component analysis (PCA), can be found in step b) of the method according to the present invention.

c) Module for Inputting the Features into a Predictive Model

In accordance with another embodiment of the system according to the present invention, in module c), the features can be inputted into a predictive model, which is built and fitted by using one or more machine learning techniques.

In accordance with another embodiment of the method according to the present invention, in module c), the predictive model can be built and fitted by using one or more machine learning techniques selected from the group consisting of Ridge Regression, Bayesian Regression, Supporting Vector Machine (SVM), k-Nearest Neighbours (k-NN) Regression, Decision Tree, and Gaussian Process Regression.

In accordance with another embodiment of the method according to the present invention, in module c), the predictive model can be built and fitted by using Bayesian regression.

In accordance with another embodiment of the method according to the present invention, in module c), hyperparameter tuning can be performed for the predictive model.

In accordance with another embodiment of the method according to the present invention, in module c), the predictive model can be fitted by a dataset of the features including a formulation type, a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, a ratio of absorbing type UV filter vs scattering/reflecting type UV filter, a SPF in vivo and a UVA-PF in vitro.

The technical details for the six machine learning techniques as well as hyperparameter tuning can be found in step c) of the method according to the present invention.

### d) Module for Calculating the UV Protection Prediction Value of the Sunscreen Product

In accordance with another embodiment of the system according to the present invention, in module d), the UV protection prediction value of the sunscreen product can be calculated by the predictive model of module c).

In accordance with another embodiment of the method according to the present invention, in module d), the UV protection prediction value can be selected from the group consisting of SPF in vivo and UVA-PF in vitro.

The prediction accuracy results can be found in step d) of the method according to the present invention.

While certain embodiments have been described, these embodiments have been presented by way of example only, and are not intended to limit the scope of the inventions. The attached claims and their equivalents are intended to cover all the modifications, substitutions and changes as would fall within the scope and spirit of the invention.

1.-18. (canceled)

19. A method for UV protection prediction of a sunscreen product, the method comprising the following steps:
a) selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter;
c) inputting the features into a predictive model, which is built and fitted by using one or more machine learning techniques; and
d) calculating the UV protection prediction value of the sunscreen product by the predictive model of step c).

20. The method according to claim 19, characterized in that the method further comprises a step b) for transforming the features of step a) by performing one or more dimensionality reduction techniques, between step a) and step c).

21. The method according to claim 20, characterized in that in step b), one or more dimensionality reduction techniques selected from the group consisting of Principal Component Analysis (PCA), Linear Discriminant Analysis (LDA), and Kernel Principal Component Analysis (KPCA) are performed.

22. The method according to claim 20, characterized in that in step b), Principal Component Analysis (PCA) is performed to obtain four principal components of the features.

23. The method according to claim 19, characterized in that in step c), the predictive model is built and fitted by using one or more machine learning techniques selected from the group consisting of Ridge Regression, Bayesian Regression, Supporting Vector Machine (SVM), k-Nearest Neighbours (k-NN) Regression, Decision Tree, and Gaussian Process Regression.

24. The method according to claim 19, characterized in that in step c), the predictive model is built and fitted by using Bayesian regression.

25. The method according to claim 19, characterized in that in that in step c), hyperparameter tuning is performed for the predictive model.

26. The method according to claim 19, characterized in that in step c), the predictive model is fitted by a dataset of the features including a formulation type, a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, a ratio of absorbing type UV filter vs scattering/reflecting type UV filter, a SPF in vivo and a UVA-PF in vitro.

27. The method according to claim 19, characterized in that the UV protection prediction value is selected from the group consisting of SPF in vivo and UVA-PF in vitro.

28. A system for UV protection prediction of a sunscreen product, the system including the following modules:
a) a module for selecting the features of the sunscreen product, wherein the features include a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, and a ratio of absorbing type UV filter vs scattering/reflecting type UV filter;
c) a module for inputting the features into a predictive model, which is built and fitted by using one or more machine learning techniques; and
d) a module for calculating the UV protection prediction value of the sunscreen product by the predictive model of module c).

29. The system according to claim 28, characterized in that the system further includes a module b) for transforming the features of module a) by performing one or more dimensionality reduction techniques.

30. The system according to claim 29, characterized in that in module b), one or more dimensionality reduction techniques selected from the group consisting of Principal Component Analysis (PCA), Linear Discriminant Analysis (LDA), and Kernel Principal Component Analysis (KPCA) are performed.

31. The system according to claim 29, characterized in that in module b), Principal Component Analysis (PCA) is performed to obtain four principal components of the features.

32. The system according to claim 28, characterized in that in module c), the predictive model is built and fitted by using one or more machine learning techniques selected from the group consisting of Ridge Regression, Bayesian Regression, Supporting Vector Machine (SVM), k-Nearest Neighbours (k-NN) Regression, Decision Tree, and Gaussian Process Regression.

33. The system according to claim 28, characterized in that in module c), the predictive model is built and fitted by using Bayesian regression.

34. The system according to claim 28, characterized in that in module c), hyperparameter tuning is performed for the predictive model.

35. The system according to claim 28, characterized in that in module c), the predictive model is fitted by a dataset of the features including a formulation type, a viscosity, a high polarity emollient, a medium polarity emollient, a low polarity emollient, a UVA filter, a UVB filter, a ratio of UVB filter vs UVA filter, a ratio of UV filter in oil phase vs UV filter in water phase, a ratio of absorbing type UV filter vs scattering/reflecting type UV filter, a SPF in vivo and a UVA-PF in vitro.

36. The system according to claim 28, characterized in that the UV protection prediction value is selected from the group consisting of SPF in vivo and UVA-PF in vitro.
